(19) United States
(12) Patent Application Publication
(10) Pub. No.: US 2002/0032183 A1
(43) Pub. Date: Mar. 14, 2002

(54) Use of (di-substituted-phenyl)-pyrimidinyl-imidazole derivatives as JNK-inhibitors

(76) Inventors: Philip LoGrasso, Carlsbad, CA (US); JeanMarie Lisnock-Geissler, Middletown, NJ (US); Steven Xanthoudakis, Beaconsfied (CA); John Tam, Montreal (CA); Sarah J. Harper, Bishop Stortford (GB); James G. Bilsland, Bishop's Storford Hertfordshire (GB); Lisa Young, Harlow (GB)

Correspondence Address: MERCK AND CO INC, P O BOX 2000, RAHWAY, NJ, 070650907

(21) Appl. No.: 09/864,949

(22) Filed: Sep. 17, 2001

Related U.S. Application Data
(60) Provisional application No. 60/208,718, filed on Jun. 1, 2000.

(51) Int. Cl. A61K 31/554; A61K 31/553; A61K 31/55; A61K 31/5377; A61K 31/541; A61K 31/506

(52) U.S. Cl. 514/210.2; 514/256; 514/217.06; 514/211.15; 514/227.8; 514/235.8

(57) ABSTRACT

A method of promoting neuronal survival and helping prevent neuronal death administers (di-substituted-phenyl) pyrimidinyl imidazole derivative compounds represented by

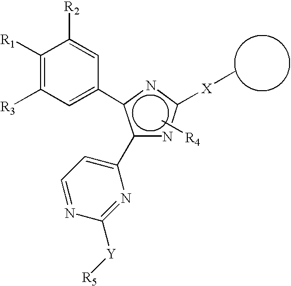

effective to inhibit the activity of c-jun-N-terminal kinase.

### BACKGROUND OF THE INVENTION

1. Field of the Invention

The present invention is directed to a method of use of (di-substituted-phenyl) pyrimidinyl imidazole derivative compounds to inhibit c-jun-N-terminal kinase. In particular, this invention is directed to a method of use of (di-substituted-phenyl) pyrimidinyl imidazole derivative compounds to promote neuronal survival and help prevent neuronal death by inhibiting c-jun-N-terminal kinase.

2. Related Background

Extracellular stimuli can cause a wide range of responses from the cell receiving such stimuli. One common response is the expression by the cell of specific proteins functionally responsive to the stimulus. There are, however, many intermediate steps between a stimulus and the resulting responsive expression of protein. The stimulus/response processes typically follow pathways (cascades) that are mediated at each step by enzymes, the presence of which facilitates the step. Conversely, the absence of a mediating enzyme can suppress the step, thereby suppressing the response.

Humans are composed of cells and some cellular responses can cause problems for people. For example, neuronal death can result from apoptosis caused by a cellular response to stress. Thus, it would be desirable to provide a method of preventing neuronal death and promote neuronal survival by inhibiting a cellular response detrimental to neurons.

As described in, for example, Y. T. Ip and R. J. Davis, Curr. Opin. Cell Biol., 10:205-219 (1998) and A. Minden and M. Karin, Biochimica et Biophysica Acta, 1333:F85-F104 (1997), certain stimuli that include stress, UV radiation, and cytokines can initiate a cascade which leads to the phosphorylation of the transcriptional activation domains of the transcription factor c-Jun. The phosphorylation of c-Jun is mediated by c-Jun N-terminal kinase (“JNK”) which is a mitogen-activated protein kinase (“MAP kinase” or “MAPK”). The transcription factor c-Jun has been implicated in cell proliferation, cell differentiation, and neoplastic transformation. It has been speculated that JNK might play a role in cellular apoptosis. Thus, it would be desirable to provide a method of preventing cellular apoptosis by inhibiting the appropriate MAP kinase that mediates the apoptosis cellular response.

U.S. Pat. Nos. 5,736,381 and 5,804,427 describe cytokine, stress, and oncoprotein activated human kinase kinases. U.S. Pat. Nos. 5,717,100, 5,859,041, 5,783,664, 5,955,366, UK Patent Publication GB 2 336 362, and International Patent Publication WO 99/47512, WO 97/33883, and WO 98/24782 describe various methods of treatment by the inhibition of cytokines and compounds that inhibit cytokines. The compounds utilized by the method of the present invention are described in U.S. Pat. No. 5,859,041. However, cytokine stimulus can produce responses other than neuronal distress, such as inflammation. Further, as described above, neuronal distress can result from celluar responses to stimuli other than cytokines. Thus, it would be desirable to provide a method of preventing neuronal distress by inhibiting the appropriate MAP kinase further downstream from the stimulus and more proximate to the response detremental to neurons. Such a method can provide better specificity with fewer unwanted side effects.

### BRIEF DESCRIPTION OF THE FIGURES

FIG. 1 is a graphical plot of the % inhibition vs. concentration of an Example of the invention.

### SUMMARY OF THE INVENTION

The present invention promotes neuronal survival by an administration of an amount of a compound represented by Formula (I), or a pharmaceutically acceptable salt thereof, effective to inhibit the activity of c-jun-N-terminal kinase:

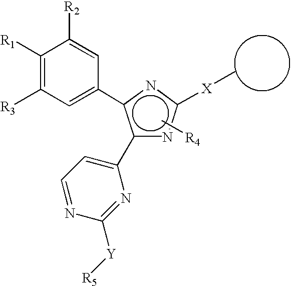

### DETAILED DESCRIPTION OF THE INVENTION

A method of this invention promotes neuronal survival by an administration of an amount of a compound represented by Formula (I), or a pharmaceutically acceptable salt thereof, effective to inhibit the activity of c-jun-N-terminal kinase:

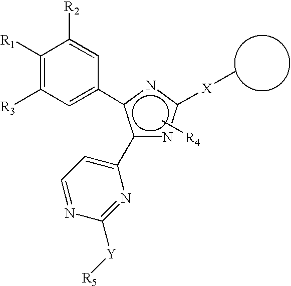

wherein

R₁ is —F, —Cl, —Br, —OH, —SH, —NH₂, or —CH₃;

R₂ is —F, —Cl, —Br, —OH, —SH, —NH₂, or —CH₃;

R₃ is —H, —F, —Cl, —Br, —OH, —SH, —NH₂, —CH₃, —OCH₃, or —CH₂CH₃;

R₄ is —C₁₋₄alkyl optionally substituted with a —C₃₋₇cycloalkyl;

R₅ is —C₁₋₄alkyl or —C₃₋₇cycloalkyl, wherein the —C₁₋₄alkyl is optionally substituted with a phenyl;

X is a bond or an alkyl bridge having 1-3 carbons;

Y is —NH— or —NH₂⁺—; and

HETCy is a 4 to 10 membered non-aromatic heterocycle containing at least one N atom, optionally containing 1-2 additional N atoms and 0-1 O or S atom, and optionally substituted with —C₁₋₄alkyl or —C(O)—O—CH₂phenyl.

In one aspect, a method of this invention administers an amount of a compound represented by Formula (I), or a pharmaceutically acceptable salt thereof, effective to inhibit the activity of c-jun-N-terminal kinase, wherein

R₁ is —Cl;

R₂ is —F, —Cl, —Br, —OH, —SH, —NH₂, or —CH₃;

R₃ is —H, —F, —Cl, —Br, —OH, —SH, —NH₂, —CH₃, —OCH₃, or —CH₂CH₃;

R₄ is —C₁₋₄alkyl optionally substituted with a —C₃₋₇cycloalkyl;

R₅ is —C₁₋₄alkyl or —C₃₋₇cycloalkyl, wherein the —C₁₋₄alkyl is optionally substituted with a phenyl;

X is a bond or an alkyl bridge having 1-3 carbons;

Y is —NH— or —NH₂⁺—; and

HETCy is a 4 to 10 membered non-aromatic heterocycle containing at least one N atom, optionally containing 1-2 additional N atoms and 0-1 O or S atom, and optionally substituted with —C₁₋₄alkyl or —C(O)—O—CH₂phenyl.

In an embodiment of this aspect, a method of this invention administers an amount of a compound represented by Formula (I), or a pharmaceutically acceptable salt thereof, effective to inhibit the activity of c-jun-N-terminal kinase, wherein

R₁ is —Cl;

R₂ is —F, —Cl, —Br, —OH, —SH, —NH₂, or —CH₃;

R₃ is —H;

R₄ is —C₁₋₄alkyl optionally substituted with a —C₃₋₇cycloalkyl;

R₅ is —C₁₋₄alkyl or —C₃₋₇cycloalkyl, wherein the —C₁₋₄alkyl is optionally substituted with a phenyl;

X is a bond or an alkyl bridge having 1-3 carbons;

Y is —NH— or —NH₂⁺—; and

HETCy is a 4 to 10 membered non-aromatic heterocycle containing at least one N atom, optionally containing 1-2 additional N atoms and 0-1 O or S atom, and optionally substituted with —C₁₋₄alkyl or —C(O)—O—CH₂phenyl.

In another embodiment of this aspect, a method of this invention administers an amount of a compound represented by Formula (I), or a pharmaceutically acceptable salt thereof, effective to inhibit the activity of c-jun-N-terminal kinase, wherein

R₁ is —Cl;

R₂ is —Cl;

R₃ is —H;

R₄ is —C₁₋₄alkyl optionally substituted with a —C₃₋₇cycloalkyl;

R₅ is —C₁₋₄alkyl or —C₃₋₇cycloalkyl, wherein the —C₁₋₄alkyl is optionally substituted with a phenyl;

X is a bond or an alkyl bridge having 1-3 carbons;

Y is —NH— or —NH₂⁺—; and

HETCy is a 4 to 10 membered non-aromatic heterocycle containing at least one N atom, optionally containing 1-2 additional N atoms and 0-1 O or S atom, and optionally substituted with —C₁₋₄alkyl or —C(O)—O—CH₂phenyl.

In a second aspect, a method of this invention administers an amount of a compound represented by Formula (I), or a pharmaceutically acceptable salt thereof, effective to inhibit the activity of c-jun-N-terminal kinase, wherein

R₁ is —F, —Cl, —Br, —OH, —SH, —NH₂, or —CH₃;

R₂ is —F, —Cl, —Br, —OH, —SH, —NH₂, or —CH₃;

R₃ is —H, —F, —Cl, —Br, —OH, —SH, —NH₂, —CH₃, —OCH₃, or —CH₂CH₃;

R₄ is —C₁₋₄alkyl optionally substituted with a —C₃₋₇cycloalkyl;

R₅ is —C₁₋₄alkyl or —C₃₋₇cycloalkyl, wherein the —C₁₋₄alkyl is optionally substituted with a phenyl;

X is a bond;

Y is —NH— or —NH₂⁺—; and

HETCy is a 4 to 10 membered non-aromatic heterocycle containing at least one N atom, optionally containing 1-2 additional N atoms and 0-1 O or S atom, and optionally substituted with —C₁₋₄alkyl or —C(O)—O—CH₂phenyl.

In an embodiment of this aspect, a method of this invention administers an amount of a compound represented by Formula (I), or a pharmaceutically acceptable salt thereof, effective to inhibit the activity of c-jun-N-terminal kinase, wherein

R₁ is —F, —Cl, —Br, —OH, —SH, —NH₂, or —CH₃;

R₂ is —F, —Cl, —Br, —OH, —SH, —NH₂, or —CH₃;

R₃ is —H, —F, —Cl, —Br, —OH, —SH, —NH₂, —CH₃, —OCH₃, or —CH₂CH₃;

R₄ is —C₁₋₄alkyl optionally substituted with a —C₃₋₇cycloalkyl;

R₅ is —C_{1 4}alkyl or —C₃₋₇cycloalkyl, wherein the —C₁₋₄alkyl is optionally substituted with a phenyl;

X is a bond;

Y is —NH—; and

HETCy is a 4 to 10 membered non-aromatic heterocycle containing at least one N atom, optionally containing 1-2 additional N atoms and 0-1 O or S atom, and optionally substituted with —C₁₋₄alkyl or —C(O)—O—CH₂phenyl.

In another embodiment of this aspect, a method of this invention administers an amount of a compound represented by Formula (I), or a pharmaceutically acceptable salt thereof, effective to inhibit the activity of c-jun-N-terminal kinase, wherein

R₁ is —F, —Cl, —Br, —OH, —SH, —NH₂, or —CH₃;

R₂ is —F, —Cl, —Br, —OH, —SH, —NH₂, or —CH₃;

R₃ is —H, —F, —Cl, —Br, —OH, —SH, —NH₂, —CH₃, —OCH₃, or —CH₂CH₃;

R₄ is —C₁₋₄alkyl optionally substituted with a —C₃₋₇cycloalkyl;

R₅ is —C₁₋₄alkyl, optionally substituted with a phenyl;

X is a bond;

Y is —NH—; and

HETCy is a 4 to 10 membered non-aromatic heterocycle containing at least one N atom, optionally containing 1-2 additional N atoms and 0-1 O or S atom, and optionally substituted with —C₁₋₄alkyl or —C(O)—O—CH₂phenyl.

In still another embodiment of this aspect, a method of this invention administers an amount of a compound represented by Formula (I), or a pharmaceutically acceptable salt thereof, effective to inhibit the activity of c-jun-N-terminal kinase, wherein

R₁ is —F, —Cl, —Br, —OH, —SH, —NH₂, or —CH₃;

R₂ is —F, —Cl, —Br, —OH, —SH, —NH₂, or —CH₃;

R₃ is —H, —F, —Cl, —Br, —OH, —SH, —NH₂, —CH₃, —OCH₃, or —CH₂CH₃;

R₄ is —C₁₋₄alkyl optionally substituted with a —C₃₋₇cycloalkyl;

R₅ is —C₃cycloalkyl;

X is a bond;

Y is —NH—; and

HETCy is a 4 to 10 membered non-aromatic heterocycle containing at least one N atom, optionally containing 1-2 additional N atoms and 0-1 O or S atom, and optionally substituted with —C₁₋₄alkyl or —C(O)—O—CH₂phenyl.

In yet another embodiment of this aspect, a method of this invention administers an amount of a compound represented by Formula (I), or a pharmaceutically acceptable salt thereof, effective to inhibit the activity of c-jun-N-terminal kinase, wherein

R₁ is —F, —Cl, —Br, —OH, —SH, —NH₂, or —CH₃;

R₂ is —F, —Cl, —Br, —OH, —SH, —NH₂, or —CH₃;

R₃ is —H, —F, —Cl, —Br, —OH, —SH, —NH₂, —CH₃, —OCH₃, or —CH₂CH₃;

R₄ is —C₁₋₄alkyl optionally substituted with a —C₃₋₇cycloalkyl;

R₅ is —C₆cycloalkyl;

X is a bond;

Y is —NH—; and

HETCy is a 4 to 10 membered non-aromatic heterocycle containing at least one N atom, optionally containing 1-2 additional N atoms and 0-1 O or S atom, and optionally substituted with —C₁₋₄alkyl or —C(O)—O—CH₂phenyl.

In another embodiment of this aspect, a method of this invention administers an amount of a compound represented by Formula (I), or a pharmaceutically acceptable salt thereof, effective to inhibit the activity of c-jun-N-terminal kinase, wherein

R₁ is —F, —Cl, —Br, —OH, —SH, —NH₂, or —CH₃;

R₂ is —F, —Cl, —Br, —OH, —SH, —NH₂, or —CH₃;

R₃ is —H, —F, —Cl, —Br, —OH, —SH, —NH₂, —CH₃, —OCH₃, or —CH₂CH₃;

R₄ is —C₁₋₄alkyl optionally substituted with a —C₃₋₇cycloalkyl;

R₅ is —C₃cycloalkyl;

X is a bond;

Y is —NH₂⁺—; and

HETCy is a 4 to 10 membered non-aromatic heterocycle containing at least one N atom, optionally containing 1-2 additional N atoms and 0-1 O or S atom, and optionally substituted with —C₁₋₄alkyl or —C(O)—O—CH₂phenyl.

In another aspect, a method of this invention administers an amount of an amine bis trifluoroacetic acid salt of a compound represented by Formula (I).

The method of this invention utilizes a subset of compounds of particular interest described by Formula (I) wherein HETCy represents a 5-6 membered non-aromatic heterocycle with 1-2 nitrogen atoms contained therein. In this subset, HETCy is advantageously a pyrrolidinyl or piperidinyl group, and particularly advantageously a 4-piperidinyl group. Within this subset of compounds, all other variables are as described previously.

As used herein, “alkyl” as well as other groups having the prefix “alk” such as, for example, alkoxy, alkanoyl, alkenyl, alkynyl and the like, means carbon chains which may be linear or branched or combinations thereof. Examples of alkyl groups include methyl, ethyl, propyl, isopropyl, butyl, sec- and tert-butyl, pentyl, hexyl, heptyl and the like. “Alkenyl”, “alkynyl” and other like terms include carbon chains containing at least one unsaturated C-C bond.

The term “cycloalkyl” means carbocycles containing no heteroatoms, and includes mono-, bi- and tricyclic saturated carbocycles. Examples of cycloalkyl include cyclopropyl, cyclobutyl, cyclopentyl, cyclohexyl, and the like.

The term “C₀₋₆alkyl” includes alkyls containing 6, 5, 4, 3, 2, 1, or no carbon atoms. An alkyl with no carbon atoms is a hydrogen atom substituent.

The term “hetero” unless specifically stated otherwise includes one or more O, S, or N atoms. For example, heterocycloalkyl and heteroaryl include ring systems that contain one or more O, S, or N atoms in the ring, including mixtures of such atoms. The hetero atoms replace ring carbon atoms. Thus, for example, a heterocycloC₅alkyl is a five member ring containing from 5 to no carbon atoms.

The term “optionally substituted” is intended to include both substituted and unsubstituted. Thus, for example, optionally substituted aryl could represent a pentafluorophenyl or a phenyl ring. Further, optionally substituted multiple moieties such as, for example, alkylaryl are intended to mean that the aryl and the aryl groups are optionally substituted. If only one of the multiple moieties is optionally substituted then it will be specifically recited such as “an alkylaryl, the aryl optionally substituted with halogen or hydroxyl.”

Compounds described herein contain one or more double bonds and may thus give rise to cis/trans isomers as well as other conformational isomers. The method of the present invention includes the utilization of all such possible isomers as well as mixtures of such isomers.

Compounds described herein can contain one or more asymmetric centers and may thus give rise to diastereomers and optical isomers. The method of the present invention includes the utilization of all such possible diastereomers as well as their racemic mixtures, their substantially pure resolved enantiomers, all possible geometric isomers, and pharmaceutically acceptable salts thereof. The above Formula I is shown without a definitive stereochemistry at certain positions. The method of the present invention includes the utilization of all stereoisomers of Formula I and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

The compounds utilized by the method of the present invention are described in U.S. Pat. No. 5,859,041 and methods of preparation are described therein of the compounds utilized by the method of the present invention.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric, and tartaric acids.

The pharmaceutical compositions of the present invention comprise a compound represented by Formula I (or pharmaceutically acceptable salts thereof) as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

Creams, ointments, jellies, solutions, or suspensions containing the compound of Formula I can be employed for topical use. Mouth washes and gargles are included within the scope of topical use for the purposes of this invention.

Dosage levels from about 0.01 mg/kg to about 140 mg/kg of body weight per day are useful in the treatment of conditions such as stroke, Parkinsons disease, Alzheimer's disease, amyotrophiclateral sclerosis, multiple sclerosis, spinal cord injury, head trauma, and seizure which are responsive to JNK inhibition, or alternatively about 0.5 mg to about 7 g per patient per day. For example, stroke may be effectively treated by the administration of from about 0.01 mg to 50 mg of the compound per kilogram of body weight per day, or alternatively about 0.5 mg to about 3.5 g per patient per day. Further, it is understood that the JNK inhibiting compounds of this invention can be administered at prophylactically effective dosage levels to prevent the onset of symptoms associated with the above-recited conditions.

The amount of active ingredient that may be combined with the carrier materials to produce a single dosage form will vary depending upon the host treated and the particular mode of administration. For example, a formulation intended for the oral administration to humans may conveniently contain from about 0.5 mg to about 5 g of active agent, compounded with an appropriate and convenient amount of carrier material which may vary from about 5 to about 95 percent of the total composition. Unit dosage forms will generally contain between from about 1 mg to about 500 mg of the active ingredient, typically 25 mg, 50 mg, 100 mg, 200 mg, 300 mg, 400 mg, 500 mg, 600 mg, 800 mg or 1000 mg.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

In practice, the compounds represented by Formula I, or pharmaceutically acceptable salts thereof, utilized by the method of this invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions utilized by the method of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compound represented by Formula I, or pharmaceutically acceptable salts thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions utilized by the method of this invention may include a pharmaceutically acceptable carrier and a compound or a pharmaceutically acceptable salt of Formula I. The compounds of Formula I, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques

A tablet containing the composition utilized by the method of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.1 mg to about 500 mg of the active ingredient and each cachet or capsule preferably containing from about 0.1 mg to about 500 mg of the active ingredient.

Pharmaceutical compositions utilized by the method of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions utilized by the method of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions utilized by the method of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by Formula I of this invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions utilized by the method of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in moulds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound described by Formula I, or pharmaceutically acceptable salts thereof, may also be prepared in powder or liquid concentrate form.

The compounds and pharmaceutical compositions utilized by the method of this invention have been found to exhibit biological activity as JNK inhibitors. Accordingly, another aspect of the invention is the treatment in mammals of, for example, stroke, Parkinsons disease, Alzheimer's disease, amyotrophiclateral sclerosis, multiple sclerosis, spinal cord injury, head trauma, and seizure—maladies that are amenable to amelioration through inhibition of the activity of JNK—by the method of this invention. The term “mammals” includes humans, as well as other animals such as, for example, rats, mice, monkeys, dogs, cats, horses, pigs, and cattle. Accordingly, it is understood that the treatment of mammals other than humans is the treatment of clinical correlating afflictions to those above recited examples that are human afflictions.

### ASSAYS DEMONSTRATING BIOLOGICAL ACTIVITY

### Biochemical Determination of Inhibition of JNK3α1

Truncated JNK3α1 (amino acids 39-422) was expressed in E. coli, purified, and activated in vitro by a combination of MKK4 and MKK7 in 129 μl of a buffer containing: 25 mM HEPES (Sigma, St. Louis, Mo.) pH 7.4, 10 mM MgCl₂ (Sigma), 2 mM DTT (Sigma), 20 mM β-glycerophosphate (Sigma), 0.1 mM Na₃VO₄, 200 μM ATP (Sigma), 225 nM JNK3α1, and 100 nM MKK4+100 nM GST-MKK7 (Upstate Biotechnology, Lake Placid, N.Y.). Activation reactions were incubated at 30° C. for 2 h.

Enzyme inhibition studies were performed at 30° C. for 30 min with 0.5 μM GST-ATF2, 1 μM ATP, 1 nM activated JNK3α1 and compound ranging from 30 pM to 100 μM. Reactions were carried out in 100 μL volumes containing the final concentrations of the following: 25 mM HEPES (Sigma) pH 7.4; 10 mM MgCl₂ (Sigma); 2 mM DTT (Sigma); 20 mM β-glycerophosphate (Sigma); 0.1 mM Na₃VO₄ (Sigma); 2 μCi [γ-³³P]ATP (2000 Ci/mmol; 1 Ci=37 GBq) (Amersham Pharmacia Biotech, Piscataway, N.J.). Reactions were stopped with 100 μL 100 mM EDTA/15 mM sodium pyrophosphate. Immobilon™-P 96-well plates (Millipore MAIPNOB™ 10, available from the Millipore Corp., Bedford, Mass.) were pretreated with 100 μL methanol, followed by 100 μL 15 mM sodium pyrophosphate. Fifty μL of the stopped reaction was spotted in triplicate on the Immobilon™-P 96-well plate. The samples were vacuum-filtered and washed three times each with 100 μL 75 mM H₃PO₄ to remove unincorporated [γ-³³P]ATP. After the third H₃PO₄ wash and a final filtration step to remove H₃PO₄, 50 μL of Microscint™-20 (Packard BioScience Ltd., Pangbourne, Berkshire, U.K.) was added to each well and samples were analyzed on a Packard Topcount™ liquid scintillation counter. IC₅₀ values were determined by fitting the data to the equation for a four parameter logistic.

TABLE 1

| Compound | IC₅₀(nM) |
|---|---|
| Example 1 | 8 |
| Example 2 | 7 |
| Example 3 | 48 |
| Example 4 | 1 |
| Example 5a | 40 |
| Example 6a | 45 |
| Example 7 | 4 |

Effects of Jnk Inhibition on Dopaminergic Neuronal Survival, In Vitro Following Treatment with the Neurotoxin MPP⁺.

Methods

1. Preparation of Mesencephalic Dopaminergic Neurons.

This protocol produces a yield of dopaminergic neurons of around 0.5-1%; this is equivalent to roughly 1000-1500 dopaminergic cells in the well. 14 day gestation Sprague-Dawley rats were killed by stunning and exsanguination. Embryos were removed and decapitated, and the ventral mesencephalon dissected from the brain. The tissue was dissociated by trypsin (0.25% in Hank's BSS) digestion for 20 minutes. The trypsin was neutralized by addition of an excess of serum containing medium and the cells centrifuged at 1000 rpm for 10 minutes. The cell pellet was resuspended in DMEM/10% FCS, and a single cell suspension prepared by mechanical dissociation and passage through a 70 μm cell strainer. Trypan blue excluding cells were counted in a haemocytometer, and cells were plated into poly-D-lysine treated 8-well chamber slides at a density of 2×10⁵ cells/well in Dulbecco's MEM supplemented with 10% FCS. Cultures were incubated for 24 hours at 37° C./5% CO₂, then the medium was replaced with DMEM supplemented with SATO (final concentration; 4.3 mg/ml BSA, 0.77 μg/ml progesterone, 20 μg/ml putrescine, 0.49 μg/ml L-thyroxine, 0.048 μg/ml selenium and 0.42 μg/ml tri-iodo-thyronine).

Cultures were incubated for 5 days, then removed from the incubator and treated with compounds. Jnk inhibitors were added at concentrations ranging from 1 nM to 1 μM to 4 independent wells per concentration. 15 minutes following addition of Jnk inhibitors, MPP⁺ was added directly to the wells to give a final concentration in the well of 10 μM. 4 wells were treated with MPP⁺ 10 μM alone, and 4 left as untreated controls. zVAD-fmk 300 μM and Example 2 500 nM were used as positive controls. Once compounds and MPP⁺ had been added, cultures were returned to the incubator at 37° C./5% CO₂ for a further 48 hours prior to fixation and immunostaining.

2. Determination of TH-Immunoreactive Cell Survival

To determine the numbers of surviving dopaminergic neurons, immunocytochemistry was carried out using a rabbit polyclonal antibody raised against TH. Non-specific binding sites were blocked using 10% normal goat serum in PBS, then primary antibody was added at 4° C. overnight. The next day, the cells were washed and treated with biotin conjugated goat anti-rabbit IgG for one hour, followed by peroxidase conjugated avidin biotin complex, both made up from the Vectastain® Elite ABC kit (Vector Laboratories, Burlingame, Calif.) according to the manufacturer's instructions. Staining was visualized using Vector™ SG (Vector Laboratories) insoluble peroxidase substrate according to the manufacturer's instructions. Following staining, the gaskets were removed from the chamber slides, and the slides mounted using aqueous mountant. Slides were blinded by another investigator before quantification of TH-immunoreactive cell survival. To determine TH-immunoreactive cell survival, cells were visualized using transmitted light on a Zeiss Axiovert inverted microscope using a 10X objective. Counts were made of all the TH-immunoreactive cells present in each well.

3. Statistical Analyses

Data analysis was performed using a one way analysis of variance, followed by Dunnett's t-test. In each case the data refer to one representative experiment, with four independent replicates for each data point. Significance was reached at p<0.05. Data shown are normalized to percentage of control response; all statistical analyses, however, were carried out on the cell counts.

Results

Effects of Example 2

Table 2 shows the effects of Example 2 on survival of mesencephalic dopaminergic neurones exposed to MPP⁺. Example 2 causes a maximal effect at 500 nM, where survival is restored to 72% of untreated control. Non-specific toxicity is observed with 10 μM treatment. Significant increases are observed with concentrations of 10 nM and above; at 10 μM, however, there is a significant decrease through non-specific toxicity (*p<0.05, **p<0.01). The results shown here are the mean±standard error margin of three independent experiments.

TABLE 2

Example 2

| Treatment | Mean | SEM |
|---|---|---|
| Control | 100.00 | 2.98 |
| MPP⁺ 10 μM | 48.77 | 1.17 |
| 1 nM | 54.72 | 1.85 |
| 10 nM | 60.77* | 2.45 |
| 100 nM | 59.95* | 1.51 |
| 500 nM | 72.41** | 0.77 |
| 1 μM | 71.14** | 2.86 |
| 10 μM | 32.08 | 3.41 |

Effects of JNK Inhibition on Survival of Rat Superior Cervical Ganglion Neurons.

Summary

Rat superior cervical ganglion (sympathetic) neurons are a population of NGF dependent neurons, which die by apoptosis when deprived of NGF. Activation of c-jun-N-terminal kinase (JNK) has been implicated in apoptosis in sympathetic neurons. An inhibitor of JNK, Example 2, was tested for neuroprotective effects in two models of sympathetic neuronal cell death.

In the first model, ganglia were dissociated and plated directly into culture plates in the presence of compound for 48 hours, then fixed and survival assayed using an ELISA for GAP-43. This model will hereafter be referred to as the ‘survival assay’.

In the second model, ganglia were dissociated and plated in the presence of NGF 25 ng/ml for 4 days. The NGF was then removed by washing and application of a blocking antibody, and L-790,984 coadministered for 72 hours. Survival was then assayed using the GAP-43 ELISA. This model will be referred to hereafter as the ‘NGF deprivation assay’.

At least three experiments were carried out for each model. The results of the ELISA were verified by immunostaining cultures from one sample experiment and counting surviving sympathetic neurons. In both of these models of sympathetic neuronal apoptosis, inhibition of JNK using Example 2 resulted in a significant increase in sympathetic neuronal survival.

Methods

1. Preparation of Superior Cervical Ganglion Neurones

Superior cervical ganglia were dissected from 1-3 day old Sprague-Dawley rat neonates. Ganglia were enzymatically dissociated using 0.25% trypsin for 45 minutes. The trypsin was then inhibited using Dulbecco's MEM (DMEM) supplemented with 10% fetal bovine serum, and the cells mechanically triturated using a pipette tip to form a single cell suspension. Neurons in the suspension were counted using a haemocytometer, and plated at a density of 3000-5000 neurons per well in poly-D-lysine and laminin coated 96 well tissue culture clusters in DMEM supplemented with B27 serum substitute. Cultures were then incubated at 37° C./5% CO₂. One hour following plating, cultures were either treated with NGF 25 ng/ml or with L-790,984 at a range of concentrations for the NGF deprivation and survival assays respectively.

Cultures for the NGF deprivation assay were returned to the incubator for 4 days. Following this, the medium was aspirated, plates washed once with DMEM/B27, and the cultures treated with Example 2 at concentrations ranging from 1 nM to 10 μM, together with an anti-NGF blocking antibody at 250 ng/ml. Cultures were then returned to the incubator for a further 72 hours prior to fixation and survival quantification. Cultures treated with Example 2 immediately for the survival assay were returned to the incubator for 48 hours; cultures were then fixed and survival quantified using the GAP-43 ELISA.

2. GAP-43 ELISA Protocol

Cultures were fixed by the addition of an equal volume 4% paraformaldehyde to each well for 10 minutes; this was then aspirated, and replaced by a further volume of 4% paraformaldehyde for a further 20 minutes at room temperature. Plates were then washed three times with PBS/0.3% TX100, and non-specific binding sites blocked by the addition of 5% normal horse serum (NHS) in PBS/0.3% TX100. Plates were incubated at room temperature for one hour, then the blocking serum was aspirated without washing and replaced with primary antibody. The primary antibody used was a mouse monoclonal antibody raised against Growth Associated Protein 43 (Sigma), prepared at a dilution of 1:500 in PBS/0.3% TX100/5% NHS. Primary antibody was added to all sample wells, with four control wells returned to blocking serum to act as minus primary control. Plates were then refrigerated overnight at 4° C. The next day, plates were washed three times with PBS/0.3 TX100 and secondary antibody added. The secondary antibody used was peroxidase conjugated sheep anti-mouse IgG, and was added at a dilution of 1:1000 in PBS/0.3% TX100/5% NHS. Plates were incubated for 1 hour at room temperature, then washed three times with PBS/0.3% TX100, and K-Blue insoluble peroxidase substrate added for 30 minutes at room temperature. The optical density of the plates was then read at 650 nm, and the survival of neurons calculated and expressed as percentage of the control response.

3. Visualisation of Sympathetic Neurones for Cell Counting

Cell counts were performed on one sample plate for each model of sympathetic neuronal cell death, by the addition of a tertiary antibody to the plate, followed by avidin-biotin complex and an insoluble peroxidase substrate. Following quantification of optical density, plates were washed three times in PBS/0.3% TX100, and non-specific sites blocked using PBS/0.3% TX100/5% normal rabbit serum (“NRS”). Plates were incubated for one hour at room temperature, then the blocking serum was aspirated and replaced with biotinylated rabbit anti-sheep IgG at a dilution of 1:500 in PBS/0.3% TX10O/5% NRS. Plates were incubated in this antibody for 30 minutes, then washed and treated with peroxidase conjugated avidin-biotin complex for a further 30 minutes. Plates were washed and staining visualized using Vector SG insoluble peroxidase substrate. Cell counts were made of immunostained neurons across the whole of the surface of each well of the plate to confirm the ELISA data.

4. Statistical Analyses

All statistical analyses were made using one-way analysis of variance, followed by Dunnet's t-test comparing all groups to untreated control in the case of the survival assay with no NGF exposure, and to the response to anti-NGF 250 ng/ml in the case of the NGF deprivation assay. In both cases, significance was deemed to have been reached when p<0.05. Both the ELISA data and the cell count data refer to the mean±standard error margin of one typical experiment for both assays.

Results

Example 2 was tested for survival promoting effects in both the sympathetic neuronal survival assay and the NGF withdrawal assay. In both of these models, there was a significant increase in sympathetic neuronal survival as quantified by the GAP-43 ELISA and by cell counts. As shown in Table 3 below, in the survival assay, the response was significant at concentrations of 300 nM and above as quantified by ELISA, and at concentrations of 100 nM and above as quantified by cell counts. While in the NGF deprivation assay, shown in Table 4 below, the response was significant at concentrations of 500 nM and above, as measured by both the ELISA and cell counts.

TABLE 3

| Example 2 | ELISA Data | | | Cell Counts | | |
|---|---|---|---|---|---|---|
| Concentration | Mean | | SEM | Mean | | SEM |
| Control | 149.19 | | 4.75 | 129.75 | | 17.61 |
| 0.01 μM | 112.10 | | 4.66 | 115.50 | | 9.02 |
| 0.03 μM | 99.19 | | 10.33 | 127.25 | | 14.26 |
| 0.1 μM | 131.45 | | 11.37 | 170.50 | | 13.99 |
| 0.3 μM | 209.68 | * | 22.98 | 267.50 | ** | 10.60 |
| 1 μM | 350.81 | ** | 17.94 | 363.00 | ** | 18.64 |
| 3 μM | 270.16 | ** | 13.07 | 321.00 | ** | 11.73 |
| NGF 1 ng/ml | 618.55 | | 43.73 | 587.00 | | 13.95 |

Table 3. Effects of Example 2 in the sympathetic neuronal survival assay, measured by both ELISA and cell counts. Data shown are the mean±S.E.M. of one typical experiment of three performed; the cell count and ELISA data shown are from the same experiment consisting of four independent wells per treatment group. Significant (*p<0.05, **p<0.01) increases in cell survival compared to untreated control are observed at Example 2 concentrations of 300 nM and above in both the ELISA and cell counts. The response declines at concentrations above 3 μM (data not shown).

TABLE 4

| Example 2 | ELISA Data | | | Cell Counts | | |
|---|---|---|---|---|---|---|
| Concentration | Mean | | SEM | Mean | | SEM |
| Anti-NGF | 40.78 | | 3.43 | 73.50 | | 3.71 |
| 250 ng/ml | | | | | | |
| 0.001 μM | 35.44 | | 2.39 | 82.00 | | 3.89 |
| 0.01 μM | 28.20 | | 1.69 | 81.00 | | 7.83 |
| 0.1 μM | 22.89 | ** | 3.72 | 53.75 | | 3.75 |
| 0.5 μM | 60.15 | ** | 3.18 | 140.25 | ** | 11.55 |
| 1 μM | 102.79 | ** | 5.46 | 200.00 | ** | 12.28 |

Table 4. Effects of Example 2 in the sympathetic neuronal NGF deprivation assay, measured by both ELISA and cell counts. Data shown are the mean±S.E.M. of one typical experiment of four performed; the cell count and ELISA data shown are from the same experiment, consisting of four independent wells per treatment group. Significant (**p<0.01) increases in cell survival over cultures treated with the anti-NGF antibody at 250 ng/ml alone are observed at Example 2 concentrations of 500 nM and 1 μM in the cell count data. In the ELISA data, significant increases are observed with Example 2 concentrations of 500 nM and 1 μM; a significant lowering was observed in the ELISA at 0.1 μM, but this effect was not significant when the cell number was quantified by cell counts.

Conclusions

The JNK inhibitor Example 2 was tested in two models of sympathetic neuronal cell death, an NGF deprivation model using a blocking antibody, and a survival model. In both of these models, significant increases in the number of surviving sympathetic neurons were observed, evaluated both by an ELISA to GAP-43, and by cell counts. JNK inhibition, therefore, protects sympathetic neurons against the apoptotic cell death induced by NGF withdrawal in this neuronal population in vitro.

### Testing of Compounds in Mouse Cerebellar Granule Neurons

Isolation of Cells:

1. Dissect out cerebella from 7-9 day old CD-I mouse pups; remove meninges.

2. Mince and dissociate with trypsin. Halt trypsinization with Dnase I and egg white trypsin inhibitor.

3. Individual cells are obtained by trituration with a pasteur pipet.

4. Cells were resuspended in cell culture media [(cMEM) E-MEM), 25 mM glucose, 10% fetal bovine serum, 2 mM glutamine, 100 μg/mL gentamycin, 25 mM KCl] and seeded at 1.2×10⁵ cells per well onto 96-well microplates pre-coated with poly-D-lysine.

5. Cultures were incubated at 37° C. in 6% CO₂, and were used for experiments on day 5-7 in vitro.

Detection of Neuronal Apoptosis

1. Replace media in column 1 with serum-free cMEM. Replace medium in columns 2-12 with serum-free cMEM with low (5 mM) K+.

2. Add drug titrations (serial diluted in DMSO; final 1% DMSO). Incubate 8 h @ 37° C.

3. Spin plate @ 1500 rpm 10 min., remove media and add lysis buffer.

4. Incubate 30 min. room temp. shaking.

5. Spin plate @ 1500 rpm 10 min., transfer supernatant to fresh plate. Store @ 4° C.

6. Transfer 5 μL supernatant and 45 μL EIA reagent to EIA strip plate (positive control standards in column 12); incubate @ room temp 2 h.

7. Wash strip plate with PBS using the plate washer.

8. Add 150 μL K-blue substrate (ELISA Technologies, Inc., Gainsville, Fla.); stop using 50 μL Red Stop. Read plate @ 650 nm.

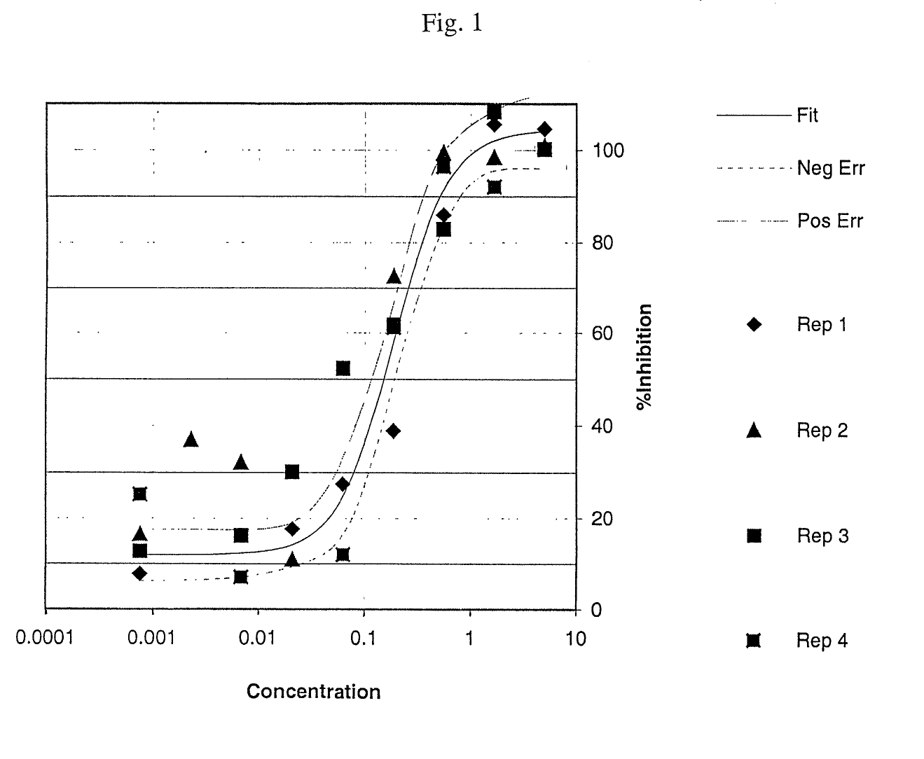

FIG. 1 shows the that the IC₅₀ of Example 2 for inhibition of neuronal apoptosis from mouse cerebellar granule neurons=100 nM.

### EXAMPLES

Compounds utilized in the method of the present invention include:

### Example 1

Cyclopropyl-{4-[5-(3,4-dichlorophenyl)-2-piperidin-4-yl-3-propyl-3H-imidazol-4-yl]-pyrimidin-2-yl}amine

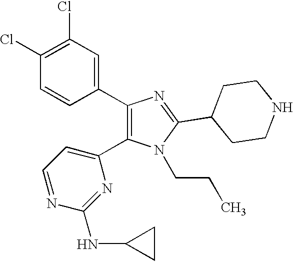

### Example 2

Cyclopropyl-{4-[5-(3,4-dichlorophenyl)-2-[(1 -methyl)-piperidin]-4-yl-3-propyl-3H-imidazol-4-yl]-pyrimidin-2-yl}amine

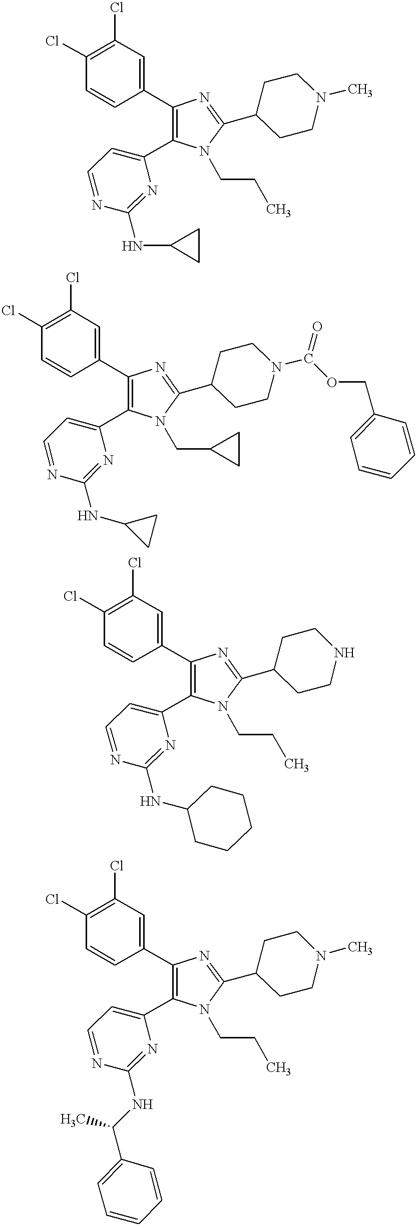

### Example 7

Cyclopropyl-{4-[3-cyclopropylmethyl-5-(3,4-dichlorophenyl)-2-piperidin-4-yl-3H-imidazol-4-yl]-pyrimidin-2-yl}amine bis trifluoroacetic acid salt

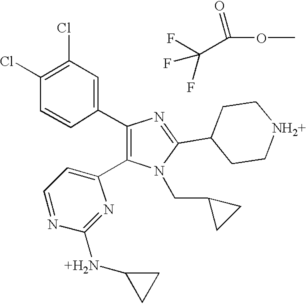

Other variations or modifications, which will be obvious to those skilled in the art, are within the scope and teachings of this invention. This invention is not to be limited except as set forth in the following claims.

What is claimed is:

1. A method of promoting neuronal survival comprising the step of administering an amount of a compound represented by Formula (I), or a pharmaceutically acceptable salt thereof, effective to inhibit the activity of c-jun-N-terminal kinase: 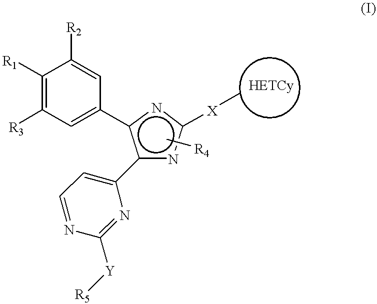
wherein
R₁ is —F, —Cl, —Br, —OH, —SH, —NH₂, or —CH₃;
R₂ is —F, —Cl, —Br, —OH, —SH, —NH₂, or —CH₃;
R₃ is —H, —F, —Cl, —Br, —OH, —SH, —NH₂, —CH₃, —OCH₃, or —CH₂CH₃;
R₄ is —C₁₋₄alkyl optionally substituted with a —C₃₋₇cycloalkyl;
R₅ is —C₁₋₄alkyl or —C₃₋₇cycloalkyl, wherein the —C₁₋₄alkyl is optionally substituted with a phenyl;
X is a bond or an alkyl bridge having 1-3 carbons;
Y is —NH— or —NH₂⁺—; and
HETCy is a 4 to 10 membered non-aromatic heterocycle containing at least one N atom, optionally containing 1-2 additional N atoms and 0-1 O or S atom, and optionally substituted with —C₁₋₄alkyl or —C(O)—O—CH₂phenyl.

2. The method according to claim 1, wherein R₁ is —Cl.

3. The method of claim 2, wherein R₃ is —H.

4. The method according to claim 1, wherein X is a bond.

5. The method of claim 4, wherein Y is —NH—.

6. The method of claim 4,
R₅ is —C₁₋₄alkyl, optionally substituted with a phenyl; and
Y is —NH—.

7. The method of claim 4, wherein
R₅ is —C₃cycloalkyl; and
Y is —NH—.

8. The method of claim 4, wherein
R₅ is —C₆cycloalkyl; and
Y is —NH—.

9. The method of claim 4, wherein
R₅ is —C₃cycloalkyl; and
Y is —NH₂⁺—.

10. The method according to claim 1, wherein said pharmaceutically acceptable salt is a bis trifluoroacetic acid salt of a compound represented by Formula (I).

11. The method according to claim 1, wherein HETCy is a 5-6 membered non-aromatic heterocycle with 1-2 nitrogen atoms contained therein.

12. The method according to claim 1, wherein said compound represented by Formula (I) is

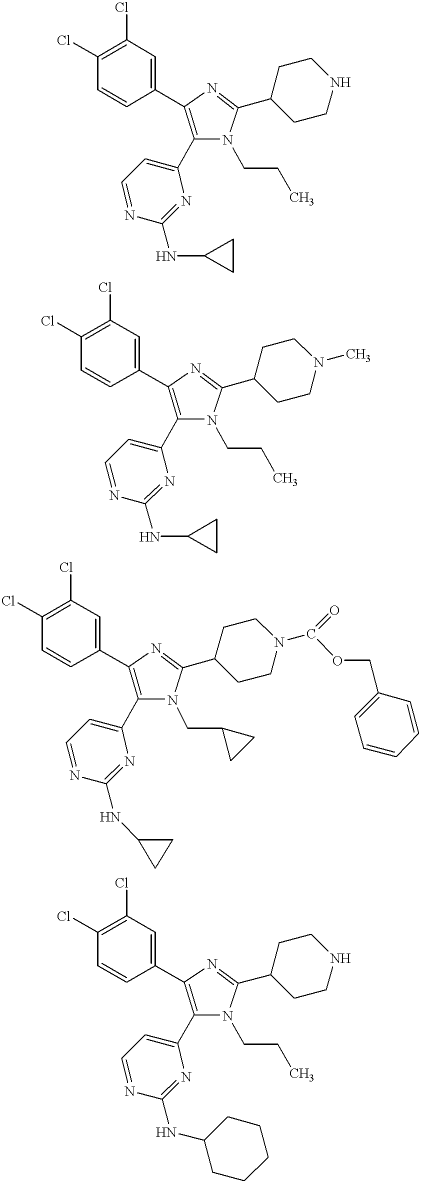

13. A method of promoting neuronal survival comprising the step of administering a therapeutic amount of a composition, said composition comprising:
a compound represented by Formula (I), or a pharmaceutically acceptable salt thereof, effective to inhibit the activity of c-jun-N-terminal kinase: 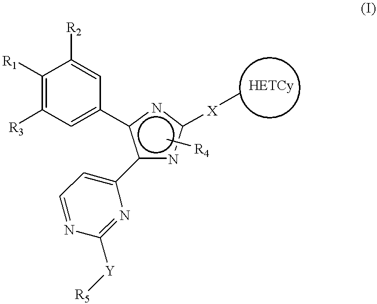
wherein
R₁ is —F, —Cl, —Br, —OH, —SH, —NH₂, or —CH₃;
R₂ is —F, —Cl, —Br, —OH, —SH, —NH₂, or —CH₃;
R₃ is —H, —F, —Cl, —Br, —OH, —SH, —NH₂, —CH₃, —OCH₃, or —CH₂CH₃;
R₄ is —C₁₋₄alkyl optionally substituted with a —C₃₋₇cycloalkyl;
R₅ is —C₁₋₄alkyl or —C₃₋₇cycloalkyl, wherein the —C₁₋₄alkyl is optionally substituted with a phenyl;
X is a bond or an alkyl bridge having 1-3 carbons;
Y is —NH— or —NH₂⁺—; and
HETCy is a 4 to 10 membered non-aromatic heterocycle containing at least one N atom, optionally containing 1-2 additional N atoms and 0-1 O or S atom, and optionally substituted with —C₁₋₄alkyl or —C(O)—O—CH₂phenyl; and
a pharmaceutically acceptable carrier.

14. A method of treatment or prevention of stroke, Parkinsons disease, Alzheimer's disease, amyotrophiclateral sclerosis, multiple sclerosis, spinal cord injury, head trauma, and seizure comprising the step of administering a therapeutically effective amount, or a prophylactically effective amount, of a compound represented by Formula (I), or a, pharmaceutically acceptable salt thereof, effective to inhibit the activity of c-jun-N-terminal kinase: 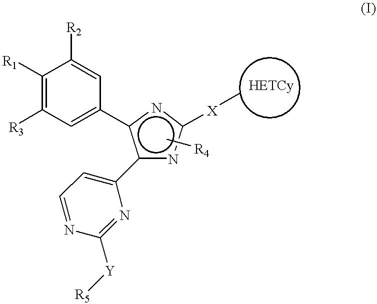
wherein
R₁ is —F, —Cl, —Br, —OH, —SH, —NH₂, or —CH₃;
R₂ is —F, —Cl, —Br, —OH, —SH, —NH₂, or —CH₃;
R₃ is —H, —F, —Cl, —Br, —OH, —SH, —NH₂, —CH₃, —OCH₃, or —CH₂CH₃;
R₄ is —C₁₋₄alkyl optionally substituted with a —C₃₋₇cycloalkyl;
R₅ is —C₁₋₄alkyl or —C₃₋₇cycloalkyl, wherein the —C₁₋₄alkyl is optionally substituted with a phenyl;
X is a bond or an alkyl bridge having 1-3 carbons;
Y is —NH— or —NH₂⁺—; and
HETCy is a 4 to 10 membered non-aromatic heterocycle containing at least one N atom, optionally containing 1-2 additional N atoms and 0-1 O or S atom, and optionally substituted with —C₁₋₄alkyl or —C(O)—O—CH₂phenyl.
